(19) United States
(12) Patent Application Publication
(10) Pub. No.: US 2025/0170191 A1
(43) Pub. Date: May 29, 2025

(54) Composition with Mitochondria-Activating Ingredient NRF, which has Telomere-Extending, Anti-Aging and Cell-Rejuvenating Function, and Method for Anti-Aging

(71) Applicant: YU-LIN YANG, Kaohsiung City (TW)

(72) Inventor: YU-LIN YANG, Kaohsiung City (TW)

(21) Appl. No.: 18/624,174

(22) Filed: Apr. 2, 2024

(30) Foreign Application Priority Data
Nov. 28, 2023 (TW) 112146090

(51) Int. Cl. A61K 35/747; A61K 35/00; A61K 35/745; A61K 36/287; A61K 36/41; A61K 36/42; A61K 36/48; A61K 36/537; A61K 36/605; A61K 36/73; A61K 36/736; A61K 45/06; A61P 39/00

(52) U.S. Cl. CPC A61K 35/747; A61K 35/745; A61K 36/287; A61K 36/41; A61K 36/42; A61K 36/48; A61K 36/537; A61K 36/605; A61K 36/73; A61K 36/736; A61K 45/06; A61P 39/00; A61K 2035/115

(57) ABSTRACT

An anti-aging composition is used to lengthen the telomere. The anti-aging composition comprises: 5-20 wt % of an extract of Salvia miltiorrhiza, 25-40 wt % of an α-amylase inhibitor and 50-60 wt % of probiotics comprising a Bifidobacterium animalis subsp. Lactis and a Lacticaseibacillus rhamnosus. The probiotics have a concentration of 1×10¹⁰CFU per gram of the anti-aging composition.

### CROSS REFERENCE TO RELATED APPLICATIONS

The application claims the benefit of Taiwan application serial No. 112146090, filed Nov. 28, 2023, and the entire contents of which are incorporated herein by reference.

### BACKGROUND OF THE INVENTION

### 1. Field of the Invention

The present invention generally relates to a composition and, more particularly, to an anti-aging composition. The present invention further relates to a method for anti-aging using the anti-aging composition.

### 2. Description of the Related Art

Aging is the process of becoming older. The term refers widely to humans, animals and fungi, etc. In general, the length of telomere is the most suitable biomarker of aging. Telomere shortening leads to the induction of chromosomal instability, and subsequently, the cell enters senescence or undergoes apoptosis.

Although telomere can be lengthened through adequate sleep, regular exercise and appropriate stress relief, no anti-aging composition exists on the market. Accordingly, an anti-aging composition and method for anti-aging using the anti-aging composition are still needed.

### SUMMARY OF THE INVENTION

It is therefore an objective of the present invention to provide an anti-aging composition to lengthening shortened telomeres.

It is another objective of the present invention to provide a method for anti-aging by using the ingredients of the anti-aging composition, effectively lengthening shortened telomeres.

As used herein, the term “a”, “an” or “one” for describing the number of the elements and members of the present invention is used for convenience, providing the general meaning of the scope of the present invention, and should be interpreted to include one or at least one. Furthermore, unless explicitly indicated otherwise, the concept of a single component also includes the case of plural components.

One embodiment of the present invention discloses an anti-aging composition can comprise 5-20 wt % of an extract of Salvia miltiorrhiza, 25-40 wt % of an α-amylase inhibitor and 50-60 wt % of probiotics. The probiotics comprise a Bifidobacterium animalis subsp. Lactis and a Lacticaseibacillus rhamnosus with a total viable count of the Bifidobacterium animalis subsp. Lactis and the Lacticaseibacillus rhamnosus per gram of the anti-aging composition being 1×10¹⁰ CFU. For example, a viable count of the Bifidobacterium animalis subsp. Lactis and the Lacticaseibacillus rhamnosus per gram of the anti-aging composition can be between 2×10⁹ and 8×10⁹ CFU, respectively, and the extract of Salvia miltiorrhiza can be obtained by extracting a root sample of Salvia miltiorrhiza by water at a temperature of from 70° C. to 100° C.

Accordingly, in the anti-aging composition according to the present invention, by the active ingredients of the extract of Salvia miltiorrhiza can activate the mitochondrial of the mammal subject, regulating the energy formation of cells. Thus, with enough energy, DNA repair and telomere maintenance in the cells can be properly processed. Moreover, by the α-amylase inhibitor, the degradation of starch ingested by the mammal subject into glucose is inhibited, thereby decreasing the absorption of sugars. By the probiotics, the microbiota in the gastrointestinal tract of the mammal subject can be regulated. By the synergistic effect of the above-mentioned components, the telomere can be lengthened, thereby achieving the therapeutic effect of anti-aging.

In the anti-aging composition according to the present invention, the anti-aging composition can further comprise 1-10 wt % of an extract selected from at least one of an extract of Medicago sativa, an extract of Momordica charantia, an extract of smoked plum, an extract of Fragaria×amamassa, an extract of Rhodiola rosea, an extract of Chrysanthemum×morifolium and an extract of leaf from Morus plant. As such, the extract of Medicago sativa can activate mitochondria together with the extract of Salvia miltiorrhiza, while the extract of Momordica charantia, the extract of smoked plum, the extract of Fragaria×amamassa, the extract of Rhodiola rosea, the extract of Chrysanthemum×morifolium and the extract of leaf from Morus plant can decrease the absorption of sugars.

Another embodiment of the present invention discloses a method for anti-aging. The anti-aging composition is administered to a mammal subject. For example, the anti-aging composition can be orally administered to the mammal subject. For instance, in the case that the mammal subject is a human subject, and for the mammal subject with body weight of 75 kg, the anti-aging composition is administered in a dosage of 4-6 g/day for 2 months. In the case that the mammal subject is a dog subject or a cat subject, for the mammal subject with body weight of 25 kg, the anti-aging composition is administered in a dosage of 1-2 g/day for 1 month.

Accordingly, by the synergistic effect of the extract of Salvia miltiorrhiza, the α-amylase inhibitor and the probiotics, the telomere can be lengthened, thereby delaying the aging of the mammal subject.

### DETAILED DESCRIPTION OF THE INVENTION

An anti-aging composition according to an embodiment of the present invention can comprise: an extract of Salvia miltiorrhiza, an α-amylase inhibitor and probiotics. By the synergistic effect of the extract of Salvia miltiorrhiza, the α-amylase inhibitor and the probiotics, the telomere can be lengthened, thereby achieving the therapeutic effect of anti-aging.

Specifically, the anti-aging composition can comprise 5-20 wt % of the extract of Salvia miltiorrhiza. Preferably, the extract of Salvia miltiorrhiza can be obtained by a method comprising the following steps: providing a sample of Salvia miltiorrhiza; extracting the sample of Salvia miltiorrhiza by water as an extractant, forming a raw extract of Salvia miltiorrhiza; and concentrating the raw extract of Salvia miltiorrhiza to obtain the extract of Salvia miltiorrhiza.

Preferably, a root sample of Salvia miltiorrhiza can be used as the sample of Salvia miltiorrhiza. Moreover, before being extracted by water, the sample of Salvia miltiorrhiza can be dried to form a dried sample of Salvia miltiorrhiza with a moisture content being equal to or smaller than 5%.

For example, for per 50 g of the sample of Salvia miltiorrhiza, the extractant of 1,000 mL can be used to mix with the sample of Salvia miltiorrhiza, forming a mixture. Then, in order to completely dissolving the active gradients of the sample of Salvia miltiorrhiza in the extractant, the sample of Salvia miltiorrhiza is extracted at a temperature of about 100° C. for a total extraction time reaching 60 minutes, forming the raw extract of Salvia miltiorrhiza, which can be appreciated by a person having ordinary skill in the art. Detail description is omitted to avoid redundancy.

After being cooled and filtrated, the raw extract of Salvia miltiorrhiza can be freeze dried to form the extract of Salvia miltiorrhiza with water content smaller than 5%. By the procedure, the active ingredients of the extract of Salvia miltiorrhiza can be concentrated, and by administration of a few of the extract of Salvia miltiorrhiza can achieve the therapeutic effect.

The anti-aging composition can further comprise 25-40 wt % of an α-amylase inhibitor. The α-amylase inhibitor can be derived from plants such as wheat (Triticum aestivum), barley (Hordeum vulgare), foxtail millet (Setaria italica), sorghum (Sorghum bicolor), peanut (Arachis hypogaea) or soybean (Glycine max). In this embodiment, the α-amylase inhibitor is derived from wheat, and has a half maximal inhibitory concentration (IC₅₀) more than or equal to 0.6 μg/g.

The anti-aging composition can also comprise 50-60 wt % of probiotics. The probiotics can comprise a Bifidobacterium animalis subsp. Lactis and a Lacticaseibacillus rhamnosus. For example, the Bifidobacterium animalis subsp. Lactis can be purchased from Bioresource Collection and Research Center with a number of BCRC 17394, BCRC 80706, BCRC 80707, BCRC 80708, BCRC 80709, BCRC80710 or BCRC 81187, etc., while the Lacticaseibacillus rhamnosus can also be purchased from Bioresource Collection and Research Center with a number of BCRC 10940, BCRC 11069, BCRC 11197, BCRC 11673, BCRC 12249, BCRC 14027, BCRC 14029, BCRC 81178, BCRC 80667, BCRC 80666, BCRC 80665, BCRC 80664, BCRC 80663, BCRC 80065, BCRC 14068, BCRC 16000, BCRC 16095, BCRC 17004, BCRC 17006, BCRC 17007 or BCRC 17490, etc. It is worthy to note that the probiotics can be live probiotics or the probiotics which are heat-inactivated (that is, the secondary metabolites of the probiotics), which can be appreciated by a person having ordinary skill in the art. Detail description is omitted to avoid redundancy.

In order to ensuring the therapeutic effect of the anti-aging composition, a total viable count of the Bifidobacterium animalis subsp. Lactis and the Lacticaseibacillus rhamnosus per gram of the anti-aging composition can be preferably equal to or more than 1×10¹⁰ CFU. For example, a viable count of the Bifidobacterium animalis subsp. Lactis and the Lacticaseibacillus rhamnosus per gram of the anti-aging composition can be between 2×10⁹ and 8×10⁹ CFU, respectively, and thus, a ratio between the Bifidobacterium animalis subsp. Lactis and the Lacticaseibacillus rhamnosus can range from 1:4 to 4:1.

Moreover, the anti-aging composition can comprise 1-10 wt % of an extract, which can be selected from at least one of an extract of Medicago sativa, an extract of Momordica charantia, an extract of smoked plum (the smoked fruit of Prunus mume), an extract of Fragaria×amamassa, an extract of Rhodiola rosea, an extract of Chrysanthemum×morifolium and an extract of leaf from Morus plant. The extract of Medicago sativa can activate mitochondria together with the extract of Salvia miltiorrhiza synergistic effect, while the extract of Momordica charantia, the extract of smoked plum, the extract of Fragaria×amamassa, the extract of Rhodiola rosea, the extract of Chrysanthemum×morifolium and the extract of leaf from Morus plant can decrease the absorption of sugars.

The anti-aging composition can be for administered to a mammal subject (e.g., the human subject belonging to primate order, or the dog subject or the cat subject belonging to carnivora order). The active ingredients of the extract of Salvia miltiorrhiza can activate the mitochondrial of the mammal subject, regulating the energy formation of cells. Thus, with enough energy, DNA repair and telomere maintenance in the cells can be properly processed. Moreover, by the α-amylase inhibitor, the degradation of starch ingested by the mammal subject into glucose is inhibited, thereby decreasing the absorption of sugars. By the probiotics, the microbiota in the gastrointestinal tract of the mammal subject can be regulated. By the synergistic effect of the above-mentioned components, the telomere can be lengthened, thereby achieving the therapeutic effect of anti-aging. For example, the anti-aging composition can be used in combination with pharmaceutical acceptable vehicles, excipients, salts or other nutrients, forming a pharmaceutical composition. In addition, the anti-aging composition can be further manufactured into any oral type that is easy to take, such as pastil, capsule, powder, pill or solution.

The anti-aging composition can be administered to the subject via any suitable routes. For example, the anti-aging composition can be orally or parenterally administrated to the subject, such as by intravenous injection (IV injection), intramuscular injection (IM injection), intraperitoneal injection (IP injection), transdermal administration, sublingual administration or nebulization administration.

Moreover, the anti-aging composition can be administered to the

mammal subject in a dosage of about 40-80 mg/kg/day. For example, for the mammal subject with body weight of 75 kg (e.g., adult male), the anti-aging composition can be administered in a dosage of 4-6 g/day for 2 months, while for the mammal subject with body weight of 25 kg (e.g., medium-sized dog), the anti-aging composition can be administered in a dosage of 1-2 g/day for 1 month. However, the dosage of the anti-aging composition may vary according to the differences of the mammal subject, the sequence of administration and the routes of administration, which can be appreciated by a person having ordinary skill in the art.

To evaluate the anti-aging composition can lengthen the telomere, the following trials are carried out:

### Trial (A)

In trial (A), a total of 20 subjects, whose ages from 28 to 67 years old, are recruited. Before the trial, the oral mucosal cells of each subject are collected, and the telomere length of the oral mucosal cells is analyzed. After orally administering the anti-aging composition for 2 months, the telomere length of the oral mucosal cells of each subject is analyzed again. The comparison results are shown in TABLE 1.

TABLE 1

| | | Telomere length (Kb) | | | Calculated age | | |
|---|---|---|---|---|---|---|---|
| | Actual | | | Vari- | | | Vari- |
| Subject | age | Before | After | ation | Before | After | ation |
| A | 52 | 1.8 | 1.9 | +0.1 | 80 | 79 | −1 |
| B | 37 | 3.1 | 3.4 | +0.3 | 68 | 65 | −3 |
| C | 35 | 3.1 | 4.5 | +1.4 | 68 | 54 | −14 |
| D | 67 | 6.1 | 7.4 | +1.3 | 38 | 25 | −13 |
| E | 51 | 4.5 | 5.9 | +1.4 | 54 | 40 | −14 |
| F | 51 | 6.5 | 7.0 | +0.5 | 34 | 30 | −4 |
| G | 47 | 4.3 | 5.1 | +0.8 | 56 | 48 | −8 |
| H | 34 | 5.3 | 8.1 | +2.8 | 46 | 18 | −28 |
| I | 65 | 2.5 | 6.9 | +4.4 | 74 | 30 | −44 |
| J | 44 | 4.2 | 5.0 | +0.8 | 57 | 49 | −8 |
| K | 55 | 2.5 | 2.9 | +0.4 | 74 | 70 | −4 |
| L | 40 | 3.5 | 3.4 | −0.1 | 64 | 65 | +1 |
| M | 35 | 3.4 | 4.5 | +1.1 | 65 | 54 | −11 |
| N | 65 | 3.5 | 3.9 | +0.4 | 64 | 60 | −4 |
| O | 28 | 8.1 | 8.2 | +0.1 | 19 | 18 | −1 |
| P | 48 | 4.4 | 4.4 | — | 55 | 55 | — |
| Q | 45 | 5.7 | 7.8 | +2.1 | 42 | 22 | −20 |
| R | 46 | 4.5 | 4.0 | −0.5 | 54 | 59 | +5 |
| S | 32 | 5.1 | 8.1 | +3.0 | 48 | 18 | −30 |
| T | 60 | 4.0 | 5.1 | +1.1 | 59 | 48 | +11 |

Referring to TABLE 1, the telomere length increases by an average of 1.074 Kb across all subjects, indicating the administration of the anti-aging composition can lengthen the telomere, and the therapeutic effect of anti-aging can be achieved.

### Trial (B)

In trial (B), a total of 9 medium-sized dogs, with their ages being from 1 to 7 years old, are recruited. The breeds of these medium-sized dogs include Labradors, wolfdogs and Giant Schnauzers. Before the trial, the oral mucosal cells of each subject are collected, and the telomere length of the oral mucosal cells is analyzed. After orally administering the anti-aging composition for 1 month, the telomere length of the oral mucosal cells of each medium-sized dog is analyzed again. The comparison results are shown in TABLE 2.

TABLE 2

| | Actual | | | Medical | Telomere length (Kb) | | |
|---|---|---|---|---|---|---|---|
| Subject | age | Gender | Breeds | history | Before | After | Variation |
| S01 | 4.0 | Male | Labrador | None | 12.29 | 25.58 | +13.29 |
| S02 | 5.0 | Male | wolfdog | None | 6.18 | 10.76 | +4.58 |
| S03 | 6.0 | Female | Labrador | None | 11.40 | 13.38 | +1.98 |
| S04 | 3.0 | Male | Labrador | None | 22.90 | 20.94 | −1.96 |
| S05 | 3.0 | Female | Labrador | None | 21.48 | 24.83 | +3.35 |
| S06 | 1.0 | Male | wolfdog | None | 18.58 | 22.34 | +3.76 |
| S07 | 7.0 | Male | Labrador | Hip | 17.89 | 18.62 | +0.73 |
| | | | | dysplasia | | | |
| S08 | 1.5 | Male | wolfdog | None | 9.39 | 20.88 | +11.49 |
| S09 | 2.0 | Male | Giant | None | 8.09 | 7.75 | −0.34 |
| | | | Schnauzer | | | | |

Referring to TABLE 2, the telomere length is increased from 14.25 Kb to 18.34 Kb, with an average of 4.09 Kb, and the p-value of the paired sample t-test is larger than 0.05, indicating the statistical significance. That is, not only in human subjects, but also in other mammal subjects, the telomeres can be lengthened by the administration of the anti-aging composition.

Accordingly, in the anti-aging composition according to the present invention, by the active ingredients of the extract of Salvia miltiorrhiza can activate the mitochondrial of the mammal subject, regulating the energy formation of cells. Thus, with enough energy, DNA repair and telomere maintenance in the cells can be properly processed. Moreover, by the α-amylase inhibitor, the degradation of starch ingested by the mammal subject into glucose is inhibited, thereby decreasing the absorption of sugars. By the probiotics, the microbiota in the gastrointestinal tract of the mammal subject can be regulated. By the synergistic effect of the above-mentioned components, the telomere can be lengthened, thereby achieving the therapeutic effect of anti-aging.

Moreover, by the synergistic effect of the extract of Salvia miltiorrhiza, the α-amylase inhibitor and the probiotics, the telomere can be lengthened, which can be confirmed though the sampling of the oral mucosal cells or the analysis of blood sample, thereby delaying the aging of the mammal subject.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. An anti-aging composition, comprising: 5-20 wt % of an extract of Salvia miltiorrhiza, 25-40 wt % of an α-amylase inhibitor and 50-60 wt % of probiotics, wherein the probiotics comprise a Bifidobacterium animalis subsp. Lactis and a Lacticaseibacillus rhamnosus, wherein a total viable count of the Bifidobacterium animalis subsp. Lactis and the Lacticaseibacillus rhamnosus per gram of the anti-aging composition is 1×10¹⁰ CFU.

2. The anti-aging composition as claimed in claim 1, wherein a viable count of the Bifidobacterium animalis subsp. Lactis and the Lacticaseibacillus rhamnosus per gram of the anti-aging composition is between 2×10⁹ and 8×10⁹ CFU, respectively.

3. The anti-aging composition as claimed in claim 1, wherein the extract of Salvia miltiorrhiza is obtained by extracting a root sample of Salvia miltiorrhiza by water at a temperature of from 70° C. to 100° C.

4. The anti-aging composition as claimed in claim 1, further comprising 1-10 wt % of an extract selected from at least one of an extract of Medicago sativa, an extract of Momordica charantia, an extract of Prunus mume, an extract of Fragaria×amamassa, an extract of Rhodiola rosea, an extract of Chrysanthemum×morifolium and an extract of leaf from Morus plant.

5. A method for anti-aging, by administrating an anti-aging composition to a mammal subject for telomere length elongation,
wherein the anti-aging composition comprises 5-20 wt % of an extract of Salvia miltiorrhiza, 25-40 wt % of an α-amylase inhibitor and 50-60 wt % of probiotics, wherein the probiotics comprise a Bifidobacterium animalis subsp. Lactis and a Lacticaseibacillus rhamnosus, wherein a total viable count of the Bifidobacterium animalis subsp. Lactis and the Lacticaseibacillus rhamnosus per gram of the anti-aging composition is 1×10¹⁰ CFU.

6. The method for anti-aging as claimed in claim 5, wherein the anti-aging composition is orally administered to the mammal subject.

7. The method for anti-aging as claimed in claim 5, wherein the mammal subject is a human subject.

8. The method for anti-aging as claimed in claim 7, wherein for the mammal subject with body weight of 75 kg, the anti-aging composition is administered in a dosage of 4-6 g/day for 2 months.

9. The method for anti-aging as claimed in claim 5, wherein the mammal subject is a dog subject or a cat subject.

10. The method for anti-aging as claimed in claim 9, wherein for the mammal subject with body weight of 25 kg, the anti-aging composition is administered in a dosage of 1-2 g/day for 1 month.
